(19) United States
(12) Patent Application Publication
(10) Pub. No.: US 2007/0219640 A1
(43) Pub. Date: Sep. 20, 2007

(54) CERAMIC-ON-CERAMIC PROSTHETIC DEVICE COUPLED TO A FLEXIBLE BONE INTERFACE

(75) Inventor: Amiram Steinberg, Avihail (IL)

Correspondence Address: JOHN SCOTT WINTERLA, KELLEY DRIVE & WARREN LLP, 400 ALTLANTIC STREET , 13TH FLOOR, STAMFORD, CT, 06901, US

(73) Assignee: Active Implants Corporation, Memphis, TN (US)

(21) Appl. No.: 11/688,153

(22) Filed: Mar. 19, 2007

Related U.S. Application Data
(60) Provisional application No. 60/743,581, filed on Mar. 20, 2006.

(30) Foreign Application Priority Data
Mar. 16, 2006 (IL) PCT/IL06/00343

(51) Int. Cl. A61F 2/32; A61F 2/34; A61F 2/46

(52) U.S. Cl. 623/22.12; 623/22.17; 606/91

(57) ABSTRACT

A prosthetic ball and socket joint comprising a ceramic on ceramic articulation surface and a resilient bone interface coupling component.

### RELATED APPLICATIONS

This application claims priority from U.S. Patent Provisional Application 60/743,581 filed Mar. 20, 2006 and PCT Application No. PCT/IL 2006/000343 filed Mar. 16, 2006.

### BACKGROUND

Joint prosthesis are used to restore near-normal function to malfunctioning natural joints. Successful prosthetic joints simultaneously satisfy several performance criteria. In addition to providing near-natural flexion between two bone structures, the articulation surfaces operate with low friction and are constructed so as to not generate any debris which could contaminate the joint. Each of the bone interfaces, of the prosthetic components, should be reliably anchored to one of the bone structures. The joint should be manufactured from materials which are stable over the life of the implant and biocompatible. By using different materials for different portions of the implant, it may be possible to simultaneously optimize the material properties for joint articulation, on the one hand, and for implant bone fixation, on the other.

### BRIEF DESCRIPTION OF DRAWINGS

FIG. 1 is a drawing of an embodiment showing an exploded view of the ceramic femoral head, the ceramic acetabular cup and the bone interface component.

FIG. 2 is a perspective drawing of an embodiment of the implantation tool.

FIG. 3 is an exploded cutaway assembly drawing of the implantation tool.

FIG. 4 is a cutaway drawing of the implantation tool head showing initial engagement of the bone interface component with the ceramic cup.

FIG. 5 is a cutaway drawing of the implantation tool head showing snap-fit engagement of the bone interface component with the ceramic cup.

### SUMMARY OF INVENTION

Embodiments herein include an articulating prosthetic ball and socket joint assembly comprising a resilient interface component manufactured from resilient material having a generally hemispherical shell shape with an inner and outer surface and a circumferential edge. The resilient interface component inner surface includes snap-fit ceramic socket liner fixation means while the outer surface includes snap-fit bone connection means. The embodiment further comprises a ceramic socket liner component having a generally hemispherical shell shape with an inner and outer surface. The ceramic socket liner component outer surface includes snap-fit resilient interface fixation means and is conformal to, and mated with, the inner surface of the resilient interface component. The ceramic socket liner is held in place by mutual engagement of the snap-fit ceramic socket liner fixation means and the snap-fit resilient interface fixation means. The embodiment also comprises ceramic ball head component having a generally spherical shape with an outer articulation surface conformal to the inner surface of the ceramic socket liner component, permitting sliding contact therewith. The ceramic ball head component further comprises a bone attachment means distal from the outer articulation surface.

### DETAILED DESCRIPTION OF INVENTION

For the purpose of this disclosure, a prosthetic hip replacement is described. It will be clear to a one of ordinary skill in the art, that the teachings are equally applicable to other articulating anatomical joints.

In an embodiment, a prosthetic replacement for a malfunctioning hip joint is disclosed. A natural hip joint is a ball and socket joint where the femur hingedly connects with the ilium. The natural femur head is a ball-like bone structure which sits in a socket-like depression in the ilium. The prosthetic joint disclosed is comprised of three functional interfaces: a ball and a mating socket which provide articulation, a stem structure for fastening the ball to the femur, and a fixation structure for connecting the socket to the ileum.

Joint tribology is determined by the properties of the contacting surfaces, the area of contact, and any lubricants associated therewith, such as synoulal fluid. The greater the area, the less the stress density applied by external loads to the joint. For a given external joint dimensional envelope, the stronger and more rigid the material employed for the socket articulating surface, the lesser the required thickness of that component. The reduction in thickness permits a corresponding increase in the diameter of the socket and an attendant increase in surface area. In addition, rigidity of the articulating surfaces can insure that contact area is not reduced by mechanical distortion resulting from asymmetric loading and other environmental conditions. For these reasons ceramic materials can be advantageously utilized for articulating surfaces.

In an embodiment, the prosthetic joint comprises a generally spherically shaped head which may be fastened to the femur using a surgically implanted stem. Alternate fastening designs may employ other structure to interconnect the head with the femur. The femoral head sits into the mating socket surgically implanted into the ilium at the location of the natural socket. Improved articulation performance may result if both the femur head and the acetabular socket articulation surfaces are ceramic. Ceramic materials can provide a strong, long-lived, low friction articulating interface when compared to other candidate materials. Ceramics, however, have properties which are not desirable for connecting to bone structures. They do not equally and adaptively distribute load stress across the bone connection surface. Additionally, being effectively non-resilient, ceramics may not provide the shock absorbing properties required for natural joint function.

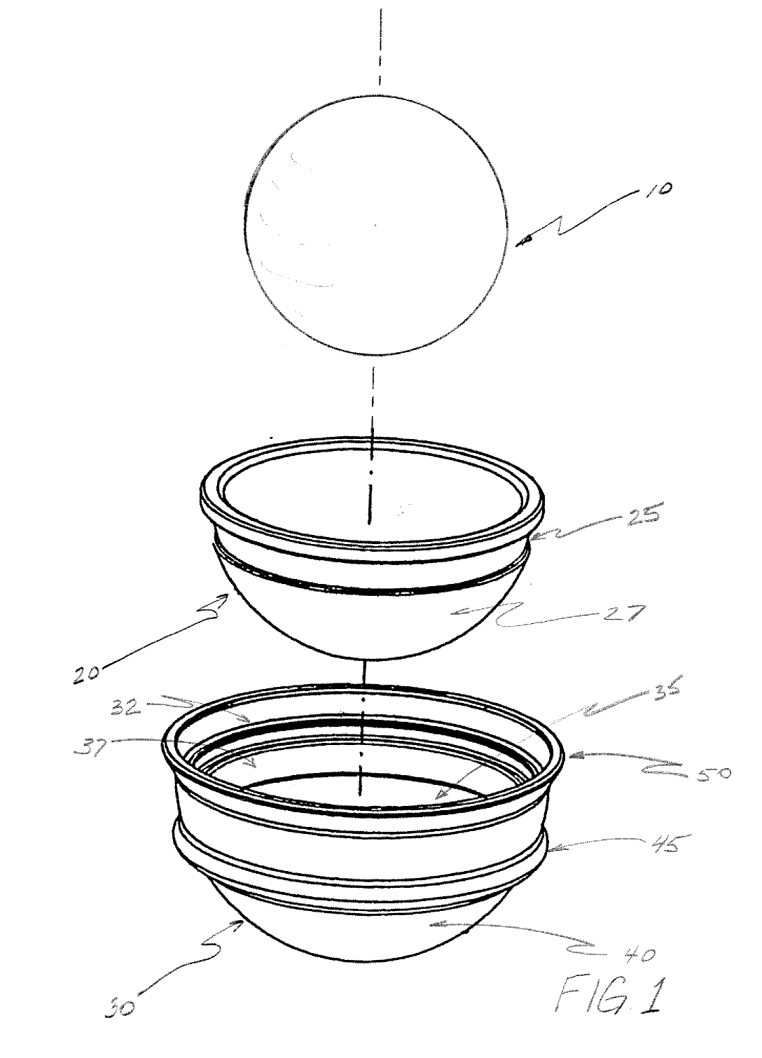

In an embodiment, a hip replacement prosthetic joint is disclosed which combines the benefits of a ceramic articulation surface with desirable load distribution and shock absorbing bone interface properties of a resilient material. Referring to FIG. 1, the prosthesis comprises three component subassemblies: the femoral head 10, the acetabular cup articulation surface 20 and the acetabular cup bone interface 30. The femoral head 10, in part, includes a fixation structure (not shown) for mechanically attaching the spherical structure to the femur. This attachment may be, for example, by way of an artificial stem surgically implanted into the longitudinal core of the femur. Alternatively, adhesive may be used to bond with the end of a suitably modified bone structure. Other methods are also suitable as would be known to one of ordinary skill in the art.

It may be desirable to make the diameter of the spherical structure of the femoral head 10 as great as possible in order to reduce load pressure. The high strength and rigidity characteristics of ceramic materials allow thickness of the articulating surface to be minimized thereby allowing the diameter to be increased for a given total joint volume. The surface properties of ceramics may also facilitate a low friction articulating surface.

The second component subassembly is the acetabular cup articulation surface 20. This component may be implemented as a hemispherical cup-like structure manufactured from a suitable ceramic material. The inside surface of the cup conformally mates with the aforementioned femoral head 10, with appropriate dimensional provision made for surface lubrication, to minimize friction. The term “conformal” is defined, for purposes of this disclosure, as a surface which engages a second surface without any significant gaps in the space between the two surfaces and with equal pressure at all points of contact. The outer surface of the cup 20 includes protrusions and/or recesses 25 for engaging the acetabular cup bone interface component 30 (the third component subassembly) utilizing a snap-fit approach. Other possible engagement mechanisms, such as, without limitation, threads or pins may alternatively be employed.

The third component subassembly, the acetabular cup bone interface component 30, is manufactured from a resilient material selected, in part, to satisfy load equalization, bio-compatibility and shock absorbing requirements. For the purpose of this disclosure, the term “resilient” is defined as having the capability to return to an original shape or position after having been compressed.

The interface component 30 is also a hemispherical shell, such shell having a greater diameter then the ceramic articulation cup 20. The inner surface 35 of the acetabular cup bone interface, which conformally engages the outer surface 27 of the ceramic acetabular articulation cup (the second component), incorporates recesses and/or protrusions 37 which are complementary to those found on the outer surface of the articulation cup. The interface component is snap-fit attached to the articulation cup by the engagement of corresponding protrusions and recesses. The outer surface 40 of the interface component, in turn, incorporates surface protrusions 45 and/or recesses which are configured to snap-fit engage complementary recesses and/or protrusions machined or otherwise fabricated into the natural bone (not shown).

The acetabular cup bone interface component 30 may also comprise of a implantation lip 50 extending from the edge 32 of the shell tangentially to the outer surface of the interface component. The lip 50 is manufactured as an integral extension of the component of the same material and has a cross section designed to be engaged by an implantation tool. During implantation, lip 50 serves as an anchoring means which, when engaged by an implantation tool, permits the application of a controlled downward force for snap-fitting the acetabular cup articulation surface 20 into the interface component 30.

Certain polyurethanes have properties which make them suitable, for the manufacture of the interface component 30 including the ability to provide adaptive load stress distribution and shock absorption.

The secure bonding of an artificial implant to the bone attachment surface may be advantageous to the long term success of the prosthesis. It is known that a bone grows or regenerates according to the stress which it must bear. In areas of high stress, bone mass will tend to increase while in areas of reduced stress, it will resorb. If the implant is exclusively manufactured from a rigid material, the bone/implant interface surface will typically be exposed to an uneven stress distribution. In accordance with Wolff's law, this situation will result in low stress areas where bone mass may be resorbed resulting in reduced contact surface area and the creation of gaps between the implant and bone surfaces. This surface area reduction, in turn, may cause increased stress to be applied at the high points resulting, concurrently, in bone growth and in excessive mechanical bone wear at those points. Each of these bone modification modes can contribute to a a failure of the attachment over time.

The optimum condition, from the bone adaptation perspective, is the uniform application of stress over the entire interface surface. This condition can not be met, over time with varying operational and environmental conditions, with a rigid prosthetic device due to lack of conformality of the bone and implant interface surfaces. What is needed is a bone interface which can adaptively and evenly redistribute the load stresses over the entire surface.

Many materials, including metals, ceramics and plastics, which have been employed in the past for implant devices are characterized, in part, by a linear stress-strain relationship. Over much of the elastic deformation range, the application of an increase of stress (force) results in a linearly proportional strain (displacement). In implants manufactured from such materials, the high points on the implant surface would experience the greatest displacement (strain) and thus produce the greatest stress. This condition would, over time, result in localized bone mass increase. In contrast, the low points on the implant surface would experience reduced strain (displacement) and thus produce reduced stress. By Wolff's law, the bone mass in this area would, over time, resorb bone material thus creating interface gaps further reducing the strain at those points. This process could potentially continue until the resorption significantly reshapes the bone interface thereby resulting in bond loosening.

Select materials that may have application herein display a stress-strain relationship which can ameliorate the shortcomings of linear stress-strain materials. The stress-strain relationship, for these select materials, is non-linear. Moreover, select materials exhibiting a stress-strain shape that can be described as a “half-bell shaped” may be employed. With respect to materials displaying a non-linear half-bell shaped curve stress-strain relationship over a portion of the elastic displacement range, the stress response to a change in displacement is significantly reduced. This “hydrostatic-like” behavior is similar to that exhibited by a non-compressible fluid. Thus, in an implant application, the stress applied across the bond surface may approach being constant regardless of surface conformity. Bone growth, in response to this condition, would be uniform across the bond surface. No localized bone resorption would result from stress applied to the bond.

In addition to the stress-strain relationship the selection of suitable materials for prosthetic applications may also satisfy other requirements. Some promising materials have been evaluated. Select polyurethanes, described below, exhibit many of the desired properties.

Bionate® (The Polymer Technology Group) is a polycarbonate-urethane (Corvita Corporation marketed it under the name Corethane® in 1996). Carbonate linkages adjacent to hydrocarbon groups give this family of materials oxidative stability, making these polymers attractive in applications where oxidation is a potential mode of degradation, such as in pacemaker leads, ventricular assist devices, catheters, and stents. Polycarbonate urethanes were among the earliest biomedical polyurethanes promoted for their biostability. Bionate® polycarbonate-urethane is a thermoplastic elastomer formed as the reaction product of a hydroxyl terminated polycarbonate, an aromatic diisocyanate, and a low molecular weight glycol used as a chain extender.

Polyurethane elastomers may be thermoplastic. Thermoplastic urethane elastomers (TPUs) combine high elongation and high tensile strength to form tough, albeit fairly high-modulus elastomers. Aromatic polyether TPUs can have advantageous flex life, tensile strength exceeding 5000 psi, and ultimate elongations greater than 700 percent. They have found use in chronic implants such as ventricular-assist devices, intraaortic balloons, and artificial heart components. TPUs can easily be processed by melting or dissolving the polymer to fabricate it into useful shapes.

The prospect of combining the biocompatibility and biostability of conventional silicone elastomers with the processability and toughness of TPUs is an attractive approach. For instance, it has been reported that silicone acts synergistically with both polycarbonate- and polyether-based polyurethanes to improve in vivo and in vitro stability. In polycarbonate-based polyurethanes, silicone copolymerization has been shown to reduce hydrolytic degradation of the carbonate linkage, whereas in polyether urethanes, the covalently bonded silicone seems to protect the polyether soft segment from oxidative degradation in vivo. PTG synthesized and patented silicone-polyurethane copolymers by combining two previously reported methods: copolymerization of silicone (PSX) together with organic (non-silicone) soft segments into the polymer backbone, and the use of surface-modifying end groups to terminate the copolymer chains.

PurSil® silicone-polyether-urethane and CarboSil® silicone-polycarbonate-urethane are thermoplastic copolymers containing silicone in the soft segment. These high-strength thermoplastic elastomers are prepared through a multi-step bulk synthesis where polydimethylsiloxane (PSX) is incorporated into the polymer soft segment with polytetramethyleneoxide (PTMO) (PurSil) or an aliphatic, hydroxyl-terminated polycarbonate (CarboSil). The hard segment consists of an aromatic diisocyanate, MDI, with a low molecular weight glycol chain extender. The copolymer chains are then terminated with silicone (or other) Surface-Modifying End Groups®.

Aromatic silicone polyetherurethanes have a higher modulus at a given shore hardness than conventional polyether urethanes—the higher the silicone content, the higher the modulus (see PurSil Properties). Conversely, the aliphatic silicone polyetherurethanes have a very low modulus and a high ultimate elongation typical of silicone homopolymers or even natural rubber (see PurSil AL Properties).

Surface Modifying End Groups® (SMEs) are surface-active oligomers covalently bonded to the base polymer during synthesis. SMEs—which include silicone (S), sulfonate (SO), fluorocarbon (F), polyethylene oxide (P), and hydrocarbon (H) groups—control surface chemistry without compromising the bulk properties of the polymer. SMEs provide a series of (biomedical) base polymers that can achieve a desired surface chemistry without the use of additives. Polyurethanes may couple endgroups to the backbone polymer during synthesis via a terminal isocyanate group, not a hard segment. The added mobility of endgroups relative to the backbone may facilitate the formation of uniform overlayersby the surface-active (end) blocks. The use of the surface active endgroups leaves the original polymer backbone intact so the polymer retains strength and processability. The fact that essentially all polymer chains carry the surface-modifying moiety eliminates many of the potential problems associated with additives. The SME approach also allows the incorporation of mixed endgroups into a single polymer. For example, the combination of hydrophobic and hydrophilic endgroups gives the polymer amphipathic characteristics in which the hydrophobic versus hydrophilic balance may be easily controlled.

CHRONOFLEX®: Biodurable Polyurethane Elastomers include polycarbonate aromatic polyurethanes (such as manufactured by CARDIOTECH CTE). The ChronoFlex® family of medical-grade segmented polyurethane elastomers have been specifically developed by CardioTech International to overcome the in vivo formation of stress-induced microfissures.

HydroThan®, Hydrophilic Thermoplastic Polyurethanes, is a family of super-adsorbent, thermoplastic, polyurethane hydrogels ranging in water content from 5 to 25% by weight, HydroThane® is offered as a clear resin in durometer hardness of 80A and 93 Shore A and is manufactured by CARDIOTECH CTE. The outstanding characteristic of this family of materials is the ability to rapidly absorb water, high tensile strength, and high elongation. The result is a polymer having some lubricious characteristics, as well as being inherently bacterial resistant due to their exceptionally high water content at the surface. HydroThane® hydrophilic polyurethane resins are thermoplastic hydrogels, and can be extruded or molded by conventional means. Traditional hydrogels on the other hand are thermosets and difficult to process.

Tecothane® (aromatic polyether-based polyurethane), Carbothane® (aliphatic polycarbonate-based polyurethane), Tecophilic® (high moisture absorption aliphatic polyether-based polyurethane) and Tecoplast® (aromatic polyether-based polyurethane) are manufactured by THERMEDICS.

Polyurethanes are designated aromatic or aliphatic on the basis of the chemical nature of the diisocyanate component in their formulation. Tecoflex, Tecophilic and Carbothane resins are manufactured using the aliphatic compound, hydrogenated methylene diisocyanate (HMDI). Tecothane and Tecoplast resins use the aromatic compound methylene diisocyanate (MDI). All the formulations, with the exception of Carbothane, are formulated using polytetramethylene ether glycol (PTMEG) and 1,4 butanediol chain extender. Carbothane is specifically formulated with a polycarbonate diol (PCDO). These represent the major chemical composition differences among the various families. Aromatic and aliphatic polyurethanes share similar properties that make them outstanding materials for use in medical devices. In general, there is not much difference between medical grade aliphatic and aromatic polyurethanes with regard to the following chemical, mechanical and biological properties: 1) High tensile strength (4,000 10,000 psi); 2) High ultimate elongation (250 700%); 3) Wide range of durometer (72 Shore A to 84 Shore D); 4) Good biocompatibility; 4) High abrasion resistance; 5) Good hydrolytic stability; 6) Can be sterilized with ethylene oxide and gamma irradiation; 7) Retention of elastomeric properties at low temperature; 8) Good melt processing characteristics for extrusion, injection molding, etc.

With such an impressive array of desirable features, it is no wonder that both aliphatic and aromatic polyurethanes have become increasingly the material of choice in the design of medical grade components. There are, however, distinct differences between these two families of polyurethane that could dictate the selection of one over the other for a particular application.

With respect to yellowing, in their natural states, both aromatic and aliphatic polyurethanes are clear to very light yellow in color. Aromatics, however, can turn dark yellow to amber as a result of melt processing or sterilization, or even with age. Although the primary objection to the discoloration of aromatic clear tubing or injection molded parts is aesthetic, the yellowing, which is caused by the formation of a chromophore in the NMI portion of the polymer, does not appear to affect other physical properties of the material. Radiopaque grades of Tecothane also exhibit some discoloration during melt processing or sterilization. However, both standard and custom compounded radiopaque grades of Tecothane have been specifically formulated to minimize this discoloration.

With respect to solvent resistance, aromatic polyurethanes exhibit better resistance to organic solvents and oils than do aliphatics—especially as compared with low durometer (80 85 Shore A) aliphatic, where prolonged contact can lead to swelling of the polymer and short-term contact can lead to surface tackiness. While these effects become less noticeable at higher durometers, aromatics exhibit little or no sensitivity upon exposure to the common organic solvents used in the health care industry.

Both aliphatic and aromatic polyether-based polyurethanes soften considerably within minutes of insertion in the body. Many device manufacturers promote this feature of their urethane products because of patient comfort advantage as well as the reduced risk of vascular trauma. However, this softening effect is less pronounced with aromatic resins than with aliphatic resins.

Tecothane, Tecoplast and Carbothane melt at temperatures considerably higher than Tecoflex and Tecophilic. Therefore, processing by either extrusion or injection molding puts more heat history into products manufactured from Tecothane, Tecoplast and Carbothane. For example, Tecoflex EG-80A and EG-60D resins mold at nozzle temperatures of approximately 310.degree. F. and 340.degree. F. respectively. Tecothane and Carbothane products of equivalent durometers mold at nozzle temperatures in the range of 380.degree. F. to 435.degree. F.

Tecoflex®, a family of aliphatic, polyether-based TPU's. These resins are easy to process find do not yellow upon aging. Solution grade versions are candidates to replace latex.

Tecothane®, a family of aromatic polyether-based TPU's available over a wide range of durometers, colors, and radiopacifiers. One can expect Tecothane resins to exhibit improved solvent resistance and biostability when compared with Tecoflex resins of equal durometers.

Carbothane®, a family of aliphatic, polycarbonate-based TPU's available over a wide range of durometers, colors, and radiopacifiers. This type of TPU has been reported to exhibit excellent oxidative stability, a property which may equate to excellent long-term biostability. This family, like Tecoflex, is easy to process and does not yellow upon aging.

Tecophilic®, a family of aliphatic, polyether-based TPU's which have been specially formulated to absorb equilibrium water contents of up to 150% of the weight of dry resin. Tecogel, a new member to the Tecophilic family, is a hydrogel that can be formulated to absorb equilibrium water contents between 500% and 2000% of the weight of dry resin. The materials were designed as a coating cast from an ethanol/water solvent system.

Tecoplast®, a family of aromatic, polyether-based TPU's formulated to produce rugged injection molded components exhibiting high durometers and heat deflection temperatures.

Elast-Eon®1, available from AorTech Biomaterials, a Polyhexamethylene oxide (PFMO), aromatic polyurethane, is an improvement on conventional polyurethane in that it has a reduced number of the susceptible chemical groups. Elast-Eon®2, a Siloxane based macrodiol, aromatic polyurethane, incorporates siloxane into the soft segment. Elast-Eon®3, a Siloxane based macrodiol, modified hard segment, aromatic polyurethane, is a variation of Elast-Eon® with further enhanced flexibility due to incorporation of siloxane into the hard segment. Elast-Eon.™ 4 is a modified aromatic hard segment polyurethane.

The Texin family is manufactured by Bayer Corporation. It comprises: 1) Texin 4210—Thermoplastic polyurethane/polycarbonate blend for injection molding and extrusion; 2) Texin 4215—Thermoplastic polyurethane/polycarbonate blend for injection molding and extrusion, 3) Texin 5250—Aromatic polyether-based medical grade with a Shore D hardness of approximately 50 for injection molding and extrusion. Complies with 21 CFR 177.1680 and 177.2600; 4) Texin 5286—Aromatic polyether-based medical grade with Shore A hardness of approximately 86 for injection molding or extrusion. Complies with 21 CFR 177.1680 and 177.2600; 5) Texin 5290—Aromatic polyether—based medical grade with a Shore A hardness of approximately 90. Complies with 21 CFR 177.1680 and 177.2600.

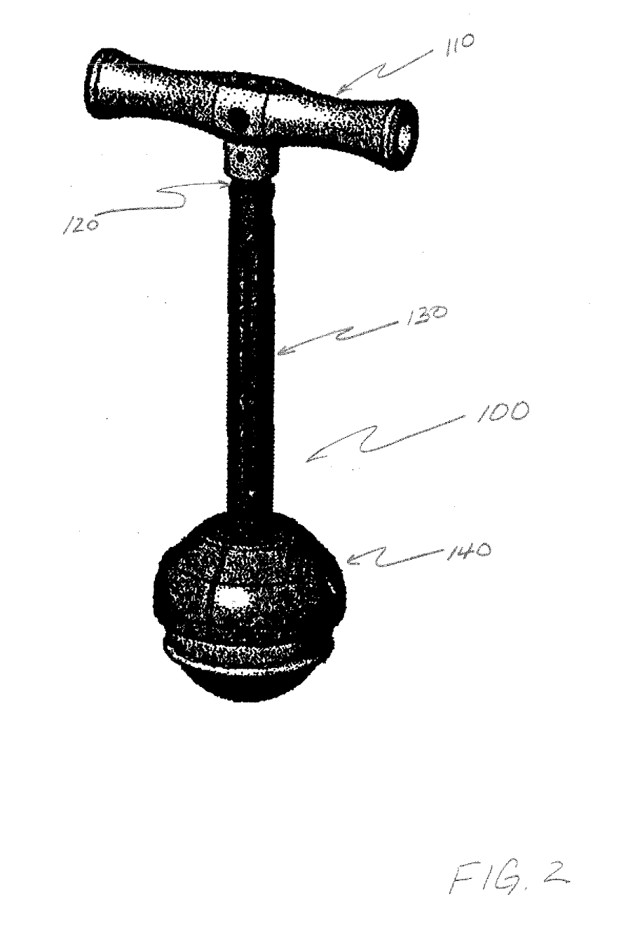

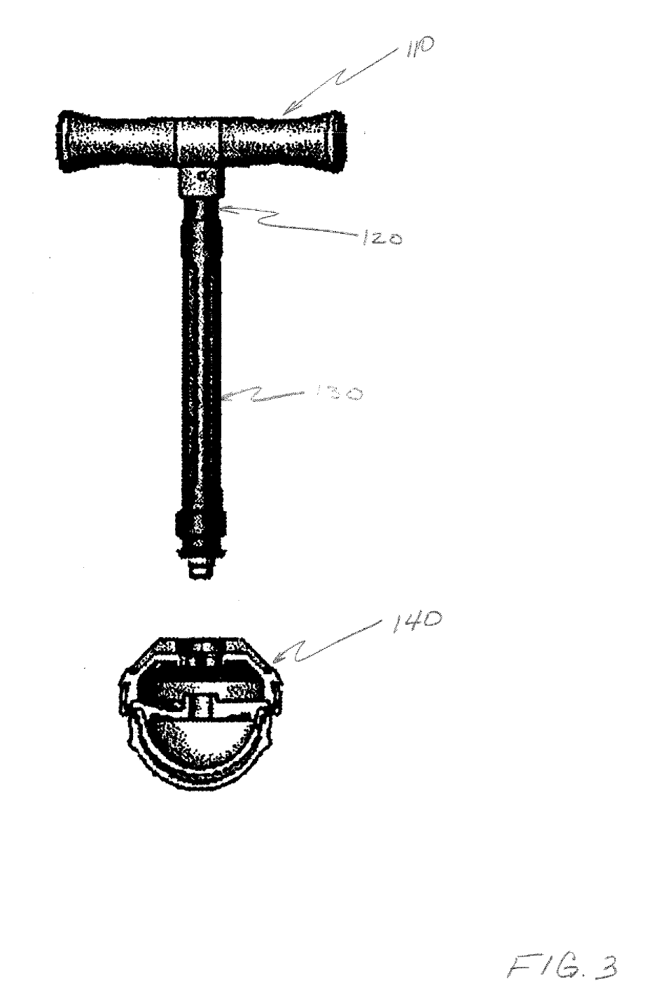

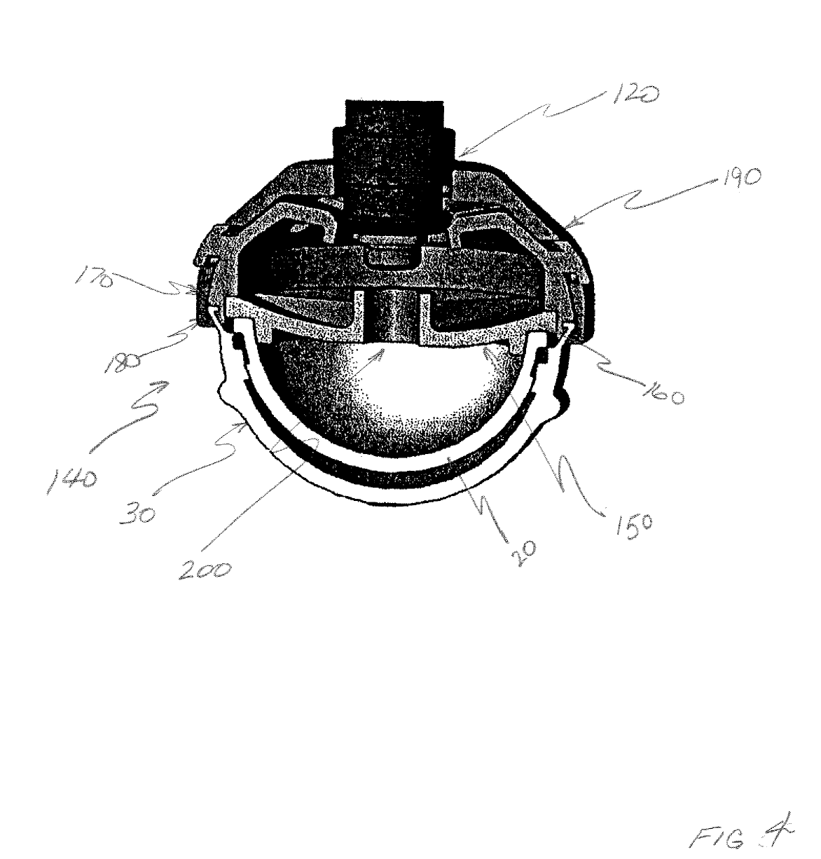

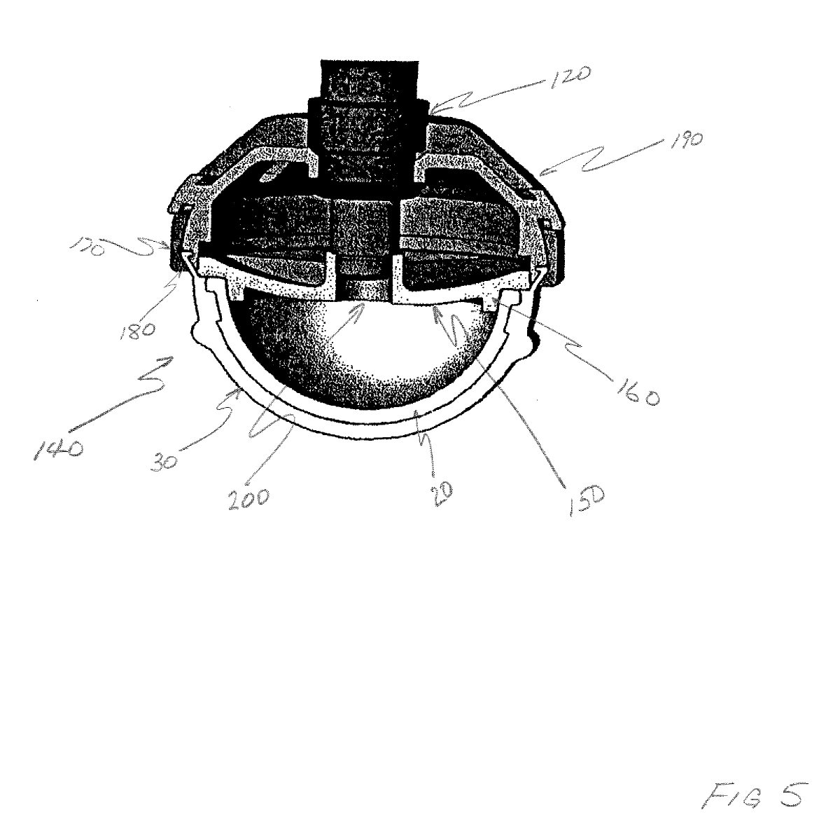

In accordance with an embodiment, the acetabular articulation surface can be implanted into the acetabular cup bone interface component in one swift, controlled, simulataneous non-impacting action utilizing a special purpose implantation tool. The natural acetabular bone surface is initially surgically modified to provide a cavity whose shape conforms to the outer surface of the bone interface component. A possible implementation of the special purpose implantation tool 100 is schematically presented in FIG. 2. In this embodiment, the tool is comprised of three major sections. Referring to FIG. 2, a handle 110 perpendicularly connected to a drive shaft 120 at its upper end allows the controlled application of downward and rotational forces. The drive shaft 120 passes through a coaxial tubular sleeve 130 which permits the shaft 120 to rotate and linearly displace along the major axis. As shown in FIG. 3, the lower end of the drive shaft 120 and the coaxial tubular sleeve 130 are connected to a generally spherically shaped implantation head 140. The implantation head 140 is shown in cutaway view in FIGS. 4 and 5 comprises an inner portion 200 and an outer portion 190. The inner portion 200 of the implantation head 140 includes a circular faced piston 150 which incorporates gripping surface 160 to retain the mouth of the ceramic articulating socket component 20. The lower end of the coaxial tubular sleeve 130 is connected to outer portion 190 of the implantation head 140. The outer portion 190 of the implantation head 140 incorporates a multi-component generally hemispheric shell 170 having a diameter comparable to that of the resilient interface component 30. The edge of the shell 170 includes slots 180 positioned to engage the mounting lip 50 of the resilient interface component 30. The initial engagement is shown in FIG. 4. Hand rotation of the coaxial tubular sleeve 130 imparts rotation on the hemispheric shell 170 causing the slots 180 to tighten their grip on the mounting lip 50. Subsequent rotation of the handle 110 causes a downward force, transmitted by the drive shaft 120, to be exerted on the held ceramic articulating socket component 20 thus snap-fitting it into the resilient interface component 30 as shown in FIG. 5. In practice, a suitable groove or other recess is reamed into the acetabular cavity so that the bone interface component 30, by virtue of its surface protrusions 45, snap-fits into the cavity. Alternatively the bone interface component may comprise a groove or recess and the bone bereamed to provide a snap-fit engaging protrusion. A subsequent rotation of the implantation tool handle 110, in the opposite direction, releases the articulation surface component 20 and the bone interface component 30. The tool 100 is then removed and the acetabular cup installation is complete.

### STATEMENT REGARDING PREFERRED EMBODIMENTS

While the invention has been described with respect to preferred embodiments, those skilled in the art will readily appreciate that various changes and/or modifications can be made to the invention without departing from the spirit or scope of the invention as defined by the appended claims. All documents cited herein are incorporated by reference herein where appropriate for teachings of additional or alternative details, features and/or technical background.

What is claimed is:

1. An articulating prosthetic ball and socket joint assembly comprising:
a resilient interface component manufactured from resilient material, said resilient interface component having a generally hemispherical shell shape with an inner and outer surface and a circumferential edge, wherein said resilient interface component inner surface includes snap-fit ceramic socket liner fixation means, said resilient interface component outer surface includes snap-fit bone connection means;
a ceramic socket liner component having a generally hemispherical shell shape with an inner and outer surface, said ceramic socket liner component outer surface including snap-fit resilient interface fixation means, said outer surface of said ceramic socket liner component being conformal to and mated with said inner surface of said resilient interface component and held in place by mutual engagement of said snap-fit ceramic socket liner fixation means and said snap-fit resilient interface fixation means;
a ceramic ball head component having a generally spherical shape with an outer articulation surface conformal to the said inner surface of the said ceramic socket liner component and permitting sliding contact therewith; said ceramic ball head component further comprising bone attachment means distal from said outer articulation surface.

2. The articulating prosthetic ball and socket joint of claim 1, wherein the said resilient interface component is manufactured from a material having a non-linear stress-strain relationship.

3. The articulating prosthetic ball and socket joint of claim 2, where the said non-linear stress-strain relationship has a “half bell-shaped” characteristic.

4. The articulating prosthetic ball and socket joint of claim 1, where said snap-fit resilient ceramic socket liner fixation means include surface protrusions and/or surface recesses.

5. The articulating prosthetic ball and socket joint of claim 1, where said snap-fit bone connection means component fixation means include surface protrusions and/or surface recesses.

6. The articulating prosthetic ball and socket joint of claim 1, where said snap-fit resilient interface component fixation means includes surface protrusions and/or surface recesses.

7. The articulating prosthetic ball and socket joint of claim 1, where said resilient interface component further comprises a mounting lip extending, tangentially to the said outer surface, from the said edge of said resilient interface component.

8. The articulating prosthetic ball and socket joint of claim 1, where said ceramic ball head component bone attachment means comprises an artificial bone stem connected to the said ceramic ball head.

9. A implantation tool for implanting a prosthetic acetabular cup comprising:
a resilient interface component manufactured from resilient material having a generally hemispherical shell shape with an inner and outer surface and an edge, said resilient interface component inner surface includes snap-fit ceramic socket liner fixation means, said resilient interface component outer surface includes snap-fit bone connection means;
said resilient interface component further comprises a mounting lip extending, tangentially to the said outer surface, from the said edge of said resilient interface component;
a ceramic socket liner component having a generally hemispherical shell shape with an inner and outer surface and a circumferential edge, said ceramic socket liner outer surface includes snap-fit resilient interface component fixation means,
said outer surface of said ceramic socket liner is conformal to and mated with said inner surface of said resilient interface component and held in place by mutual engagement of said snap-fit ceramic socket liner fixation means and said snap-fit resilient interface component fixation means;
said implantation tool comprising:
a drive shaft having a first end and a second end;
a handle having a mid-point, said mid-point fixedly and perpendicularly connected to said first end of said drive shaft;
an elongate grip, having a top end and a bottom end, rotatably and slidably co-axially mounted over said drive shaft;
a generally spherically shaped implantation head comprising an inner and an outer portion; said inner portion of said implantation head fixedly connected to said second end of said drive shaft, said inner portion of said implantation head including a circular faced piston incorporating provision for retaining said circumferential edge of said ceramic articulating socket component;
said outer portion of said implantation head fixedly connected to said bottom end of said elongate grip and including slots configured to engage said mounting lip of said resilient interface component.
